(19) United States
(12) Patent Application Publication
(10) Pub. No.: US 2026/0232194 A1
(43) Pub. Date: Aug. 13, 2026

(54) 3D quantitative amplified magnetic resonance imaging

(71) Applicants: The Board of Trustees of the Leland Stanford Junior University, Stanford, CA (US); Màtai Medical Research Institute, Gisborne (NZ)

(72) Inventors: Itamar Terem, Menlo Park, CA (US); Kawin Setsompop, Stanford, CA (US); Samantha J. Holdsworth, Gisborne (NZ)

(21) Appl. No.: 19/475,173

(22) PCT Filed: May 1, 2024
(86) PCT No.: PCT/US2024/027154

§ 371 Date: Oct. 14, 2025

Related U.S. Application Data
(60) Provisional application No. 63/463,112, filed on May 1, 2023.

(51) Int. Cl. A61B 5/00; A61B 5/055; G01R 33/561; G16H 30/40

(52) U.S. Cl. CPC A61B 5/0042; A61B 5/055; G01R 33/5614; G16H 30/40; A61B 2576/026

(57) ABSTRACT

A method for magnetic resonance imaging performs a magnetic resonance imaging (MRI) apparatus an MRI imaging scan of a brain to produce 3D MRI videos of a brain; processes the 3D MRI videos to produce a sub-voxel motion Held of the brain induced by physiological motion; and displays the sub-voxel motion field. The processing comprises: generating a 3D complex linear steerable pyramid decomposition of the 3D MRI videos into multiple scales and orientations; temporally filtering phase coefficients of the 3D complex linear steerable pyramid decomposition; and solving an optical flow equation over the phase coefficients of the 3D complex linear steerable pyramid decomposition to produce the sub-voxel motion field in the form of voxel displacement maps.

### FIELD OF THE INVENTION

The present invention relates generally to magnetic resonance imaging (MRI). More specifically, it relates to techniques to amplify and quantify in three-dimensional (3D) MRI video a sub-voxel motion field induced by physiological brain motion.

### BACKGROUND OF THE INVENTION

MRI is a noninvasive tomographic imaging technique, which produces structural and functional brain images with great details and tissue contrast. However, detecting the subtle signal changes at the mesoscopic scale (10-100 μm) with conventional MRI methods is extremely challenging due to the macroscopic image resolution (~1-2 mm) characterizing this bio-imaging modality.

Changes in blood vessel pulsation and cerebrospinal fluid dynamics cause cyclic deformation of the brain. Such brain motion can be altered by neurological pathologies such as hydrocephalus, Chiari I malformation, idiopathic intracranial hypertension, and age-related diseases in small cerebral vessels. Quantifying the cardiac- and CSF-induced (pulsatile) brain motion in vivo may therefore provide valuable insights into neurological pathologies and their effects on the brain's biomechanical response, making it a useful tool for research and has potential for assisting in the diagnosis and management of such diseases.

Various MRI techniques are available that enable one to visualize and quantify pulsatile brain motion. Phase contrast MRI (PC-MRI) uses bipolar gradients to measure tissue velocity over the cardiac cycle in different directions, which can lead to longer scanning times. To estimate the displacement, the measured velocities need to be integrated over time-which can introduce additional errors. Complementary Spatial Modulation of Magnetization (CSPAMM) measures tissue displacement by tagging or nulling the signal in the region of interest. This technique has demonstrated an ability to measure brain motion in the cranial caudal direction. However, due to this technique's low spatial resolution, it may pose difficulty at detecting small brain motion in the other two directions. Displacement encoding with stimulated echoes (DENSE) MRI is a technique that can measure tissue displacement with high spatial and temporal resolution and is sensitive to small displacements (0.01 mm). However, it requires encoding in different directions and suffers from a low SNR, both of which translate to long scan times.

Amplified MRI (aMRI) is a relatively new method that can visualize pulsatile brain tissue motion over the cardiac cycle by amplifying subtle temporal intensity changes in cardiac gated cine MRI data. aMRI offers several advantages over other techniques, including pronounced tissue contrast when coupled with the balanced steady-state free precession (bSSFP) MRI sequence, a short scan time because it does not require displacement encoding in multiple directions, and a higher spatial and temporal resolution compared to DENSE.

Additionally, aMRI can reveal mesoscopic brain deformation, making it a promising tool for different neurological disorders such as Chiari Malformation, aneurysms (aFlow), concussion, and acute hemorrhagic stroke. Recently, aMRI was extended to three dimensions, enabling the visualization of brain motion in all three planes (3D aMRI). This approach improves the image quality and visualization of pulsatile brain motion by capturing out-of-plane motion (third direction). The high spatial and temporal resolution of the underlying raw data cine acquisition, along with the conspicuous brain tissue and CSF contrast enabled by the underlying bSSFP sequence, allows for the observation of the brain's biomechanical response in exquisite detail. However, 3D aMRI still lacks the ability to quantify the sub-voxel motion field in physical units.

### BRIEF SUMMARY OF THE INVENTION

We developed a method called 3D quantitative amplified Magnetic Resonance Imaging (3D q-aMRI), which enables the visualization and quantification of brain tissue pulsatile motion over any periodic physiological process (e.g., cardiac, respiratory, blood pressure, EEG waves such as delta, theta, alpha) by amplifying subtle temporal intensity changes in the cine MRI data. 3D q-aMRI enjoys great tissue contrast, short scanning time (does not require encoding in multiple directions), can reveal mesoscopic brain deformation with resolutions indiscernible to the naked eye, and has higher spatial resolution compared to DENSE.

In q-aMRI we acquire short volumetric video clips of the brain and utilize spatial and temporal video processing techniques to amplify and quantify the sub-voxel motion field of the brain induced by blood pulsation cerebrospinal fluid (CSF) motion.

3D q-aMRI is a post-processing technique, which we apply to MRI data in the form of a smooth video. The data could either be synchronized with any of various periodic physiological processes to enable the visualization and quantification of the pulsatile brain motion, or could be acquired without synchronization in a way that is rapid enough to capture the effects of physiological or exogenous processes. The method will work also with and without synchronization, i.e., periodic or non-periodic motion. It suffices that the motions are sub-voxel, and that the data does not contain a significant amount of noise. The synchronization is beneficial to overcome the low temporal resolution of MRI.

The 3D q-aMRI post-processing technique is based on solving the optical flow equation over the phase coefficients of the 3D complex linear steerable pyramid decomposition. The technique was tested on a 3D digital phantom to characterize its behavior and limitations. In-vivo validation was performed to tune the hyperparameters and to test its ability to quantify the sub-voxel motion of the brain. Qualitative comparison to Phase-Contrast (PC) MRI, as an accepted gold standard, was done. Repeatability and reproducibility of 3D q-aMRI was evaluated using scan-rescan in healthy volunteers.

The 3D q-aMRI techniques improves on prior techniques, with several features including the following: To our knowledge this is the first implementation as motion-amplification visualization and motion-quantification tool for any major medical imaging modality. The implementation of this technique improves performance and ability to handle 4D input data. The technique is optimized for MRI data by optimizing scales and orientations levels of the 3D steerable pyramid, optimizing the temporal filtering/frequencies for MRI data, optimizing the variance/size of the gaussian kernel to optimize the extracted motion field. The technique is extended for compatibility with 3D volumetric images. The technique extends the 2D filter to a 3D filter to capture motion in the through-plane direction.

3D q-aMRI can be used as an early diagnostic tool for different neurological disorders including, for example, Chiari Malformation, Idiopathic Intracranial Hypertension, hydrocephalus, aneurysms, mild Traumatic Brain Injury, Alzheimers disease.

In one aspect, the invention provides a method for magnetic resonance imaging comprising: performing by a magnetic resonance imaging (MRI) apparatus an MRI imaging scan of a brain to produce 3D MRI video of the brain; and processing the 3D MRI video to amplify and quantify in the 3D MRI video a sub-voxel motion field induced by physiological brain motion by solving an optical flow equation over phase coefficients of a 3D complex linear steerable pyramid decomposition of the 3D MRI video. The 3D MRI video preferably comprises steady state free precession (SSFP) sequence MRI data. For example, the 3D MRI video may comprise 3D volumetric cardiac-gated cine MRI data, 3D bSSFP cine data, cine Gradient-Spoiled SSFP data, Cine RF Spoiled SSFP data, or SSFP-Echo data.

In a preferred implementation, phases of the 3D complex linear steerable pyramid decomposition are used to produce magnification and quantification of the 3D MRI video, wherein the phases are separated from the amplitude component, band-passed, processed using a conventional 3D aMRI technique to produce motion magnification, and processed through a quantification technique that solves the optical flow equation over the phases using a weighted least squares objective function over the various orientation and scales of the 3D steerable pyramid decomposition.

In another aspect, the invention provides A method for magnetic resonance imaging comprising: performing by a magnetic resonance imaging (MRI) apparatus an MRI imaging scan of a brain to produce 3D MRI videos of a brain; processing the 3D MRI videos to produce a sub-voxel motion field of the brain induced by physiological motion; and displaying the sub-voxel motion field. The processing comprises: generating a 3D complex linear steerable pyramid decomposition of the 3D MRI videos into multiple scales and orientations; temporally filtering phase coefficients of the 3D complex linear steerable pyramid decomposition; and solving an optical flow equation over the phase coefficients of the 3D complex linear steerable pyramid decomposition to produce the sub-voxel motion field in the form of voxel displacement maps.

Processing the 3D MRI videos may be performed by the magnetic resonance imaging (MRI) apparatus or, alternatively, by a device distinct from the magnetic resonance imaging (MRI) apparatus.

The physiological motion may be periodic physiological motion or non-periodic physiological motion. The 3D MRI videos are preferably steady state free precession (SSFP) sequence MRI data. For example, the 3D MRI videos may comprise 3D volumetric cardiac-gated cine MRI data, 3D bSSFP cine data, cine Gradient-Spoiled SSFP data, Cine RF Spoiled SSFP data, or SSFP-Echo data.

### BRIEF DESCRIPTION OF THE SEVERAL VIEWS OF THE DRAWINGS

FIG. 1 illustrates a 3D q-aMRI processing pipeline according to an embodiment of the invention.

FIG. 2A shows a 3D digital phantom that undergoes cyclic tension and compression according to an embodiment of the invention.

FIG. 2B is a graph of the error of the estimated motion field as a function of the motion magnitude in the X, Y, and Z directions, according to an embodiment of the invention.

FIG. 3 is a flowchart showing a method for validation of the 3D q-aMRI technique against the observed signal in 3D aMRI, according to an embodiment of the invention.

FIG. 4 is a grid of images that depicts the normalized temporal standard deviation maps of the amplified (3D aMRI) videos for six different pyramid levels, according to an embodiment of the invention.

FIG. 5 is a grid of images that shows the normalized temporal standard deviation maps of the amplified (3D aMRI) movies for different temporal frequency bands, according to an embodiment of the invention.

FIG. 6 is a grid of images that depicts the extracted motion field by 3D q-aMRI for varying Gaussian windows, according to an embodiment of the invention.

FIG. 7 is a grid of images that depicts the extracted motion field by 3D q-aMRI for different isotropic voxel sizes, according to an embodiment of the invention.

FIG. 8A is a grid of images that shows an image comparison between PC-MRI (top) and 3D q-aMRI (bottom) for sagittal (S/I direction=), coronal (S/I direction), and axial (L/R direction) planes, according to an embodiment of the invention.

FIG. 8B is a graph showing the voxel displacement profile (throughout the cardiac cycle) in the out-of-the-plane direction through the cerebral aqueduct as captured by 3D q-aMRI, according to an embodiment of the invention.

### DETAILED DESCRIPTION OF THE INVENTION

### Introduction

Embodiments of the present invention provide a 3D quantitative aMRI (3D q-aMRI) post-processing technique that enables one to visualize and quantify pulsatile voxel displacement brain motion by applying it to 3D MRI data. The data can work with any MRI contrast. In the description below it is illustrated with 3D bSSFP cine data, but is not limited to this. The 3D q-aMRI technique is an extension of the existing 3D-aMRI technique and is based on solving the optical flow equation over the coefficients of the 3D complex linear steerable pyramid decomposition.

### Methods

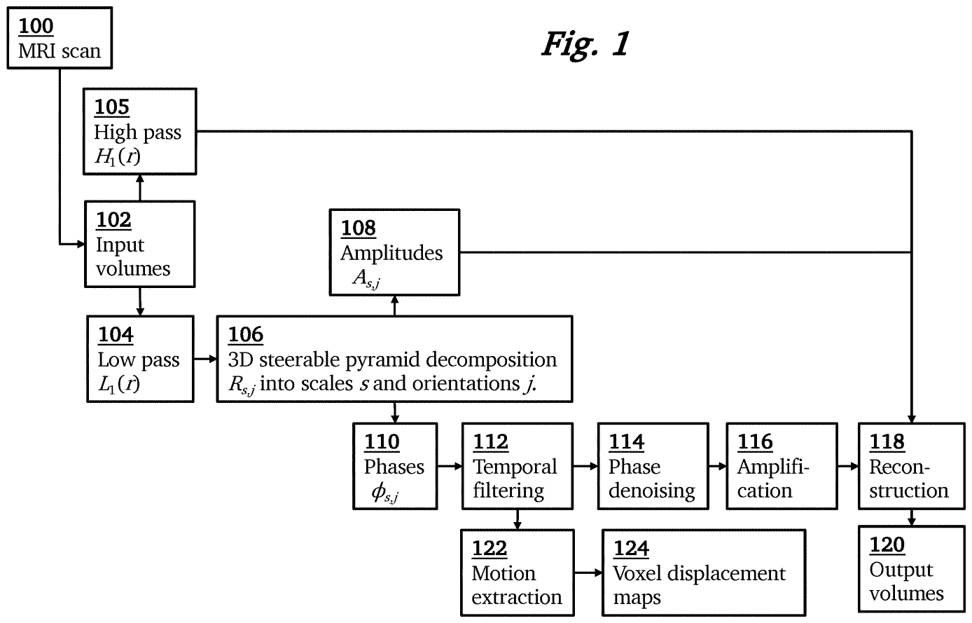

FIG. 1 illustrates a 3D q-aMRI processing pipeline according to an embodiment of the invention. An MRI scan 100 is performed to produce 3D MRI videos 102 using conventional techniques. The volumetric cine MRI data 102 is sent through a low-pass filter 104 and then decomposed by a 3D complex steerable pyramid decomposition into scales and orientations 106. The phases 110 of the decomposition are separated from the amplitude component 108. The phases 110 are independently temporally band pass filtered in a temporal filtering step 112 at each spatial location, orientation, and scale. The filtered phases are then processed in a phase denoising step 114 followed by amplification 116. The result of the amplification is combined in a reconstruction step 118 with the amplitude component 108 and high-pass filtered 105 input volumes 102 to produce output volumes 120 at the conclusion of the a visualization path. The temporally filtered phases are also split off and processed in a parallel quantification path that performs motion extraction 122 to generate voxel displacement maps 124 by solving Eq. 10 as described in more detail below.

The processing steps to generate the voxel displacement maps from the 3D MRI videos may be performed by by the MRI apparatus or, alternatively, by a separate device that the 3D MRI videos have been sent to from the MRI apparatus.

### MRI Acquisition

The MRI scan 100 was performed using a 3T MRI scanner (SIGNA Premier; GE Healthcare, Milwaukee, WI) and a 48-channel head coil. 3D volumetric cardiac-gated cine (bSSFP/FIESTA) MRI datasets 102 were acquired as follows: Sagittal plane, FOV=24×24 cm², matrix size=256×256 (upsampled), TR/TE/flip-angle=2.8 ms/1 ms/25°, acceleration factor=8, resolution=1.2 mm³ isotropic, peripheral pulse gating with retrospective binning to 20 cardiac phases, 120 slices for whole brain coverage, and a scan time of 2:30 min.

For segmentation purposes, we also collected a T1 MPRAGE volumetric data in the six volunteers who were scanned for the repeatability and reproducibility analysis. The following parameters were used: Sagittal plane, FOV=24×24 cm², matrix size=256×256 (upsampled), TR/TE/flip-angle=2500 ms/2.4 ms/8°, resolution=1.2 mm³ isotropic.

For comparison to past studies using the common phase contrast MRI (PC-MRI) approach, we also collected one set of cine PC-MRI data and 3D volumetric aMRI data on one volunteer, with the following parameters: FOV=24×24 cm², velocity encoding=1 cm/s, matrix size=256×256, TR/TE/flip-angle=50 ms/12 ms/10°. Velocity-encoding was performed in the right-left direction in the axial plane, and in the superior-inferior direction for the sagittal and coronal planes. The resulting images were masked to remove the background noise and to highlight the brain motion in these directions of interest.

### Motion Quantification

3D q-aMRI is an extension of the 3D aMRI technique. As shown in FIG. 1, the technique starts by decomposing the brain volumetric cine MRI data 102 using the 3D steerable pyramid decomposition 106. The 3D steerable pyramid is a complex linear multiscale and multi orientation decomposition in which steerable filters are used in a recursive scheme (low-pass filtering and downsampling). The scales (levels) basis functions are band pass filtered in the frequency domain. They are calculated in polar coordinates as a multiplication of low and high pass filter. The low-pass and high-pass filters for each scale are given by the following equations:

$${H}_{s} ( r ) = \{ \begin{matrix}1 , & \frac{r}{s} \geq 1 \\ \vert cos ( \frac{π}{2} {log}_{2} ( \frac{r}{s} ) ) \vert , & 0.5 > \frac{r}{s} < 1 \\ 0 , & 0 < \frac{r}{s} \leq 0.5\end{matrix} \qquad ( 1 )$$

$${L}_{s} ( r ) = \sqrt{1 - {H}_{s}^{2} ( r )} = \{ \begin{matrix}0 , & \frac{r}{s} \geq 1 \\ \vert sin ( \frac{π}{2} {log}_{2} ( \frac{r}{s} ) ) \vert , & 0.5 < \frac{r}{s} < 1 \\ 1 , & 0 < \frac{r}{s} \leq 0.5\end{matrix} \qquad ( 2 )$$

where s is the scaling factor of the level. The band pass filter of each level is given by the following,

$${B}_{s} ( r ) = {H}_{s} ( r ) \times {L}_{s - 1} ( r ) . \qquad ( 3 )$$

The angular (orientation) filters are 3D cones oriented along the six vertices of cuboctahedron (Table 1) and satisfy the following equation in the frequency domain:

$${B}_{j} ( {k}_{x} , {k}_{y} , {k}_{z} ) = \frac{{( {α}_{j} {k}_{x} + {β}_{j} {k}_{y} + {γ}_{j} {k}_{z} )}^{2}}{{k}_{x}^{2} + {k}_{y}^{2} + {k}_{z}^{2}} , j = 0 , 2 , … , 5 \qquad ( 4 )$$

where αⱼ, βⱼ, γⱼ are the direction of the axes of symmetry of the six basis filters Bⱼ.

The 3D steerable filters are constructed as following:

$${f}_{s , j} ( {k}_{x} , {k}_{y} , {k}_{z} ) = {B}_{s} ( r ) \times {B}_{j} ( {k}_{x} , {k}_{y} , {k}_{z} ) \qquad ( 5 )$$

where

$$r = \sqrt{{k}_{x}^{2} + {k}_{y}^{2} + {k}_{z}^{2}}$$

and the image response is calculated as follows:

$${R}_{s , j} ( x , y , z ) = {ℱ}^{- 1} \{ ℱ \{ I ( x , y , z ) \} \times {f}_{s , j} \} = {A}_{s , j} \cdot {e}^{i {ϕ}_{s , j}} \qquad ( 6 )$$

where [symbol] {I(x, y, z)} is the Fourier transform of the image, s and j are the scaling factor and orientation direction, respectively, and [symbol] ⁻¹ is the inverse Fourier transform.

TABLE 1
Table 1. Directions cosines for the axes of symmetry of 6 basis filters Bⱼ
with geometry based on the vertices of the cuboctahedron.

| j | αⱼ | βⱼ | γⱼ |
|---|---|---|---|
| 0 | $\frac{1}{\sqrt{2}}$ | $\frac{1}{\sqrt{2}}$ | 0 |
| 1 | $\frac{1}{\sqrt{2}}$ | $- \frac{1}{\sqrt{2}}$ | 0 |
| 2 | $\frac{1}{\sqrt{2}}$ | 0 | $\frac{1}{\sqrt{2}}$ |
| 3 | $- \frac{1}{\sqrt{2}}$ | 0 | $\frac{1}{\sqrt{2}}$ |
| 4 | 0 | $\frac{1}{\sqrt{2}}$ | $\frac{1}{\sqrt{2}}$ |
| 5 | 0 | $\frac{1}{\sqrt{2}}$ | $- \frac{1}{\sqrt{2}}$ |

The decomposition outputs a complex number with amplitude (A_{s,j}) and phase (φ_{s,j}) at each scale and orientation. The phases of the decomposition encode information about the sub-voxel motion. This information is exploited to achieve both magnification (visualization) and quantification of the voxel displacement field. The phases are separated from the amplitude component and temporally band-passed 112 to isolate the desired frequency range and to remove any DC component.

The temporally band-passed phases continue through two parallel processing path-ways. One pathway is a conventional 3D aMRI path, which includes phase denoising 114 (with amplitude-weighted Gaussian smoothing filter), amplification 116 involving multiplication by amplification factor α, and reconstruction 118 to produce motion magnification. The other pathway is a voxel displacement quantification path involving motion extraction 122, which is performed by solving Eq. 10, as described below.

The quantification technique is based on solving the optical flow equation, which assumes the brightness constancy constraint (that the brightness of a moving point in an image remains constant over time). However, there is a subtle distinction from the standard optical flow approach that we use here. The equation is instead solved over the filtered phases of the coefficients of the 3D complex-valued steerable pyramid decomposition. In the general case, the temporal evolution of (spatial) contours with constant phase in the image subbands (such as used in a complex wavelet decomposition) correspond to the motion field. Mathematically this can be written as follows:

$${ϕ}_{s , j} ( x , y , z , 0 ) = {ϕ}_{s , j} ( x - u ( x , y , z , t ) , y - v ( x , y , z , t ) , \qquad ( 7 )$$

$$z - d ( x , y , z , t ) , t ) .$$

Here φ_{s,j} (x, y, z,t) are the phases of the 3D complex steerable pyramid and (u(x, y, z, t), v(x, y, z, t), d(x, y, z, t)) are the sub-voxel displacement fields we would like to estimate. We can expand the right-hand side using the Taylor series around (x, y, z) to get the following:

$${Δϕ}_{s , j} = ( \frac{\partial {ϕ}_{s , j}}{\partial x} , \frac{\partial {ϕ}_{s , j}}{\partial y} , \frac{\partial {ϕ}_{s , j}}{\partial z} ) \cdot ( u , v , d ) + O ( {u}^{2} , {v}^{2} , {d}^{2} ) \qquad ( 8 )$$

where Δφ_{s,j}(x, y, z, t)=φ_{s,j} (x, y, z, t)−φ_{s,j} (x, y, z, 0) and O(u², v², d²) are higher order terms in the Taylor expansion. The main assumption here is that the motion is at the sub-voxel level, and as a result, the higher-order terms in the Taylor expansion are negligible. Therefore, the solution can be approximated using only the linear term:

$${Δϕ}_{s , j} = ( \frac{\partial {ϕ}_{s , j}}{\partial x} , \frac{\partial {ϕ}_{s , j}}{\partial y} , \frac{\partial {ϕ}_{s , j}}{\partial z} ) \cdot ( u , v , d ) . \qquad ( 9 )$$

This linear equation is solved in step 122 using a weighted least squares objective function over the various orientation and scales of the 3D steerable pyramid decomposition, as follows:

$${arg min}_{u , v , d} {\sum }_{W} {\sum }_{s} {\sum }_{j} {{A}_{s , j}^{2} [ ( \frac{\partial {ϕ}_{s , j}}{\partial x} , \frac{\partial {ϕ}_{s , j}}{\partial y} , \frac{\partial {ϕ}_{s , j}}{\partial z} ) \cdot ( u , v , d ) - {Δϕ}_{s , j} ]}^{2} \qquad ( 10 )$$

where W is a Gaussian window (σ=5), φ_{s,j} and A_{s,j} are the phase and amplitude of the filters's output, and Δφ_{s,j} is the phase difference between the frames tⱼ and t₀.

Notice that conventional discrete derivative operators such as the intermediate or the central kernels are sensitive to noise when estimating derivatives in high spatial frequency images, such as in the higher levels of the 3D steerable pyramid. Therefore, to increase the technique's robustness to noise and improve the estimated motion field accuracy, the phases' spatial derivatives

$$( \frac{\partial {ϕ}_{s , j}}{\partial x} , \frac{\partial {ϕ}_{s , j}}{\partial y} , \frac{\partial {ϕ}_{s , j}}{\partial z} )$$

were estimated using the following approach:

$$∇ {ϕ}_{s , j} ( \vec{x} , t ) = \frac{Im [ {R}_{s , j}^{*} ( \vec{x} , t ) \cdot ∇ {R}_{s , j} ( \vec{x} , t ) ]}{{A}_{s , j}^{2} ( \vec{x} , t )} \qquad ( 11 )$$

where R_{s,j}(x, t) is the image response for the s, j-th filter (complex number), and Im is the imaginary operator. ∇R_{s,j}(\vec{x}, t) still contains high spatial frequencies components, but it can be demodulated as follows. We can express the image response as:

$${R}_{s , j} ( \vec{x} , t ) = M ( \vec{x} , t ) \cdot {e}^{i \vec{x} {k}_{0}} \qquad ( 12 )$$

where k₀ is the peak tuning frequency of the corresponding subband's filter. Taking the derivative of R_{s,j}(\vec{x}, t) results in the following:

$$∇ {R}_{s , j} ( \vec{x} , t ) = ∇ M ( \vec{x} , t ) \cdot {e}^{i \vec{x} {k}_{0}} + {ik}_{0} \cdot {R}_{r , θ} ( \vec{x} , t ) \qquad ( 13 )$$

where M (\vec{x}, t) is the demodulated filter response, which contains only low spatial frequencies. Finally, we notice that traditional derivative filters are often inappropriate for multi-dimensional problems, therefore ∇M(\vec{x}, t) was approximated using the following derivative filters:

$$Derivative = [ 0.109604 , 0.276691 , 0 , - 0.276691 , - 0.109604 ] \qquad ( 14 )$$

$$PreFilter = [ 0.037659 , 0.249153 , 0.426375 , 0.249153 , 0.037659 ]$$

where the Derivative filter is applied in the desired direction, and the PreFilter is applied in the two remaining directions.

In comparison to the traditional intensity based optical flow techniques, which estimate motion larger than one voxel, this approach of using image local phase information can estimate sub-voxel motion in the order of 0.01 pixel size.

### Digital Phantom Simulation

To validate the developed technique, we tested 3D q-aMRI on a 3D digital phantom to assess its behavior and limitations. In vivo validation was performed to fine-tune the hyperparameters and to test the ability of the technique to quantify voxel displacement motion observed in 3D aMRI. The results from 3D q-aMRI were qualitatively compared to phase-contrast MRI (PC-MRI), the conventional technique for capturing brain motion.

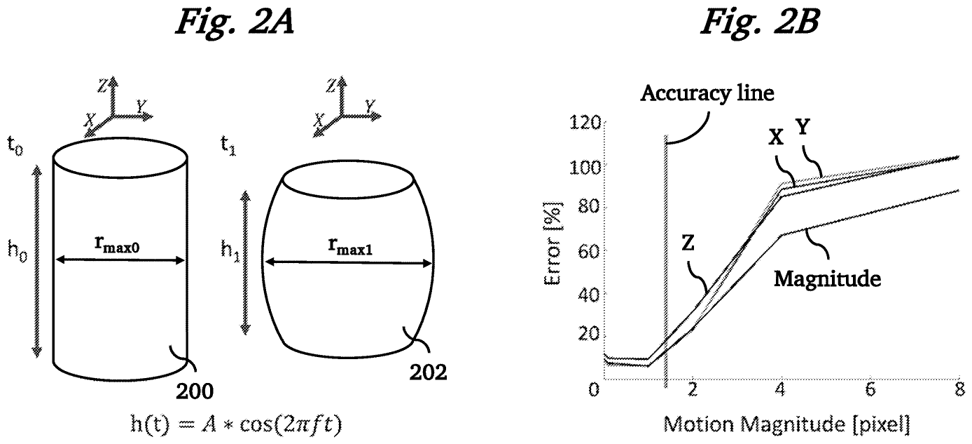

To evaluate the performance of 3D q-aMRI, a 3D digital phantom that undergoes cyclic tension and compression was used to mimic the subtle deformation, intensity, and contrast of the lateral ventricles observed in 3D aMRI. The phantom has the form of a 3D cylinder with an initial height h₀ and initial radius r₀, and was simulated using MATLAB (Math Works, Natick, MA, version 2022b). The two ends of the 3D cylinder were deformed according to the following equation: hₜ=A*cos(2πft), resulting in cyclic compression and tension along its axis. This is illustrated in FIG. 2A, which shows the phantom at reference time to 200 and at deform time t₁ 202.

We made the following assumptions:
1. The phantom volume remains constant over time, resulting in expansion and compression along the cylinder's radius.
2. The radius follows parabolic curve: r(z)=a*z²+rₘₐₓ, where rₘₐₓ is the maximum radius and z is the position along the axis of the cylinder of height hₜ. These assumptions result in the following equation relating a and rₘₐₓ:

$$\frac{- {h}_{t}^{5}}{80} {a}^{2} + \frac{{h}_{t}^{3} * {r}_{max}}{6} a - {h}_{0} * {r}_{0}^{2} = 0 \qquad ( 15 )$$

Notice that the solution for a depends on rₘₐₓ. We choose max as follows:

$${r}_{max} = \frac{{r}_{0}}{2} ( \sqrt{25 - 30 * ( 1 - \frac{{h}_{t}}{{h}_{0}} )} - 5 ) . \qquad ( 16 )$$

The estimated motion field was evaluated against the true motion field using the following metrics:
1. Pearson's Linear Correlation

$$Error [ \% ] = Mean [ abs [ \frac{{x}_{estimated} - {x}_{true}}{{x}_{true}} ] * 100 ] [ \% ] . \qquad 2$$

3. 99 percentile of the Error distribution

### In Vivo Validation

First it is important to note that we undertook the following standardization procedures:
1. Since images acquired with different native image size will determine the extent and resolution of the 3D steerable pyramid filters, we therefore ensured that all in vivo datasets were zero padded such that each dataset was 256×256×256.
2. Accordingly, for the analysis of different image resolutions, we used the corresponding σ values to match the physical extent of the Gaussian smoothing filter. For example, to compare an isotropic 2.4 mm³ (128× 128× 128) versus an isotropic 1.2 mm³ (256× 256× 256) dataset (the latter with o=5), we first zeropad the 2.4 mm³ dataset and use o=2.5 to ensure the filter has the same extent as the 1.2 mm³ dataset in physical units.
3. To generate a sharp frequency response of the temporal filter that will pass the desired frequency band, the number of cardiac phases were extended to 5 periods, which is a valid procedure since the motion of the cine data is periodic. This was performed since the native number of cardiac phases of 20 may generate side lobes in the filter frequency spectrum-which in turn may propagate errors in the voxel displacement estimation due to noise.

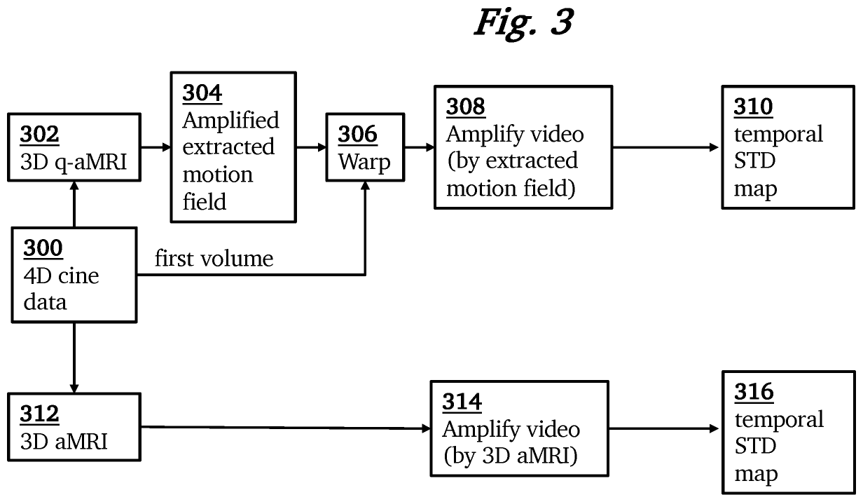

To validate the ability of 3D q-aMRI to accurately quantify the observed motion in 3D aMRI, we followed three steps. First, 3D aMRI was used to amplify the signal in the 4D in vivo data. Then, the voxel displacement field was calculated using 3D q-aMRI, and a second amplified movie was created by warping the first volume in the data according to an amplified version of the estimated motion field. Next, normalized temporal standard deviation maps were calculated for both amplified movies FIG. 3, and these were compared qualitatively. We also tested and optimized the hyperparameters (number of pyramid levels, temporal filtering, and Gaussian window σ parameter) for the in vivo data. Lastly, the effect of varying image resolution on the estimated voxel displacement field was tested.

### In Vivo Analysis and Repeatability Study

An in vivo analysis was conducted to provide insights into the performance and potential applications of the 3D q-aMRI technique in biomedical research and clinical practice. We qualitatively compared 3D q-aMRI against ground truth phase contrast MRI data. The motion profile was also evaluated in the through plane direction through the cerebral aqueduct. The repeatability and reproducibility of 3D q-aMRI were assessed using 4-scans-4-rescan (total of 8 scans) in 6 healthy volunteers. For each volunteer we conducted the following: first, the MPRAGE data was registered (rigid body registration using MATLAB 2022) to the first cine bSSFP scan. Second, the registered MPRAGE data was used to segment the following brain regions: lateral ventricles, 3rd ventricle, 4rd ventricle, brainstem and cerebellum. Next, the voxel displacement field was extracted for each scan using 3D q-aMRI. Each scan (of the eight) was registered (rigid body registration using MATLAB 2022) to the first scan, and the extracted transformation was applied to each of the voxel displacement fields. Finally, the average motion profile in the superior and inferior direction throughout the cardiac cycle was extracted for each of the brain regions. Finally, preliminary 3D q-aMRI data was acquired on patients with neurode-generative disorders and a healthy aging control to assess the potential diagnostic value in identifying disease-induced biomechanical differences.

### Results

### Phantom Simulations

The following was evaluated: (1) the accuracy as a function of the magnitude of the true motion; (2) the robustness to various Gaussian noise levels (see Table 2); and (3) the effects of the hyperparameters (number of pyramid levels in the least square, temporal filtering, and Gaussian window σ parameter) on the estimated motion field.

FIG. 2B is a graph of the error of the estimated motion field as a function of the motion magnitude in the X, Y, and Z directions, in the absence of noise. The technique is accurate for sub-voxel motion as small as 0.001 of a pixel size, but breaks down for large motions>1.5 pixels. Table 2 shows that with added Gaussian noise, 3D q-aMRI can still robustly quantify motion>0.005 of a pixel size, while staying robust to noise. Voxel displacements can be accurately extracted for an SNR as low as 25, maintaining high correlation (0.95), low error (13%), and relatively small error distribution tail (99% of the error is below 30%). Notice that these results represent an ideal limit, and that in practice there are other sources of ‘noise’ that were not simulated in the digital phantom.

We also demonstrated the effects of the hyperparameters on performance. The estimated field error as a function of the number of pyramid levels was used to solve the weighted least squares equation. The results depend on the type of motion, whether it be local deformation or global translation. For global translation, using all pyramid levels results in better accuracy, whereas for local deformation, only the first two highest levels of the pyramid are required.

We also investigated the effect of temporal filtering on the estimated motion field for an SNR of 25. Prior knowledge of the expected motion (temporal frequencies present in the data) has a significant impact on the accuracy of the estimated voxel displacement field. We utilize the fact that our data is cardiac gated and filter out temporal frequencies outside the 1-4 Hz band when estimating the voxel displacement field.

TABLE 2
Correlation, error estimation, and 99 percentiles
of the error distribution as a function of SNR
for motion magnitudes >0.005 of a pixel size.

| SNR | No noise | 200 | 100 | 50 | 25 | 12.5 | 6.25 |
|---|---|---|---|---|---|---|---|
| Correlation | 0.98 | 0.98 | 0.97 | 0.96 | 0.95 | 0.93 | 0.94 |
| Error [%] | 5.69 | 5.88 | 6.60 | 9.35 | 12.65 | 12.12 | 16.16 |
| Percentile (99%) | 18.13 | 19.18 | 19.09 | 27.31 | 30.26 | 38.81 | 48.16 |
| Sigma | 5 | 5 | 7.5 | 10 | 12.5 | 15 | 17.5 |

We also demonstrated the effect of varying the Gaussian standard deviation on the estimated motion field for an SNR of 25. Increasing the standard deviation leads to a better estimation field up to a certain threshold, after which the estimated field remains almost unchanged.

### In Vivo Validation

In vivo validation of the 3D q-aMRI technique against the observed signal in 3D aMRI is performed as illustrated in FIG. 3. The 4D cine data 300 is processed by 3D aMRI 312 to produce amplified video 314. The 4D cine data 300 is also processed by 3D q-aMRI 302 to produce amplified extracted motion field 304. The first volume in the cine data 300 is warped by an amplified version of the estimated motion field 304 to produce amplified video 308. Temporal variance maps are calculated for both amplified movies to produce temporal STD maps 310, 316. The maps show that 3D q-aMRI successfully quantified the sub-voxel motion observed in 3D aMRI.

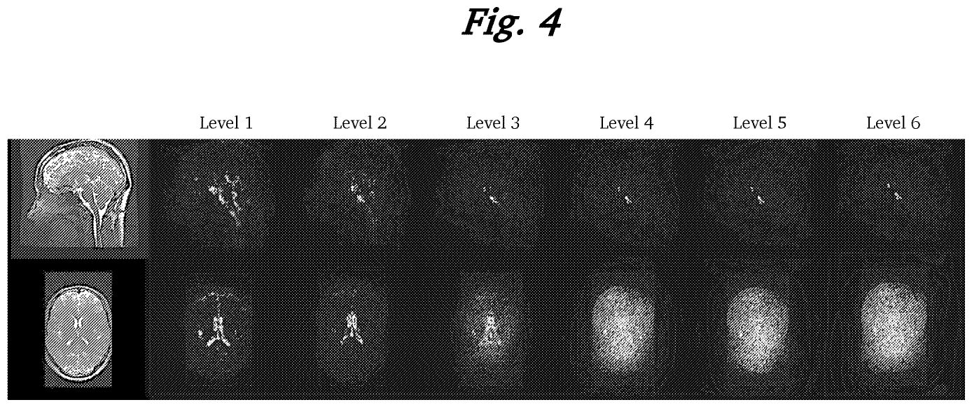

The validation procedure of FIG. 3 demonstrates that 3D q-aMRI successfully quantified the sub-voxel motion observed in 3D aMRI using the optimized hyperparameters. These parameters include band pass filtering (1-4 Hz) the phases, and solving the weighted least squares using only the first two highest levels of the 3D steerable pyramid with a Gaussian window of σ=5. Justification for these choices is provided in FIGS. 4-6.

FIG. 4 depicts the normalized temporal standard deviation maps of the amplified (3D aMRI) videos for different pyramid levels (levels 1-6). Most of the motion information is observed within the first two levels of the 3D steerable pyramid. The data was amplified with an amplification parameter of 30 with a Gaussian window with σ=5. Coherent motion exists mainly in the first two levels of the steerable pyramid.

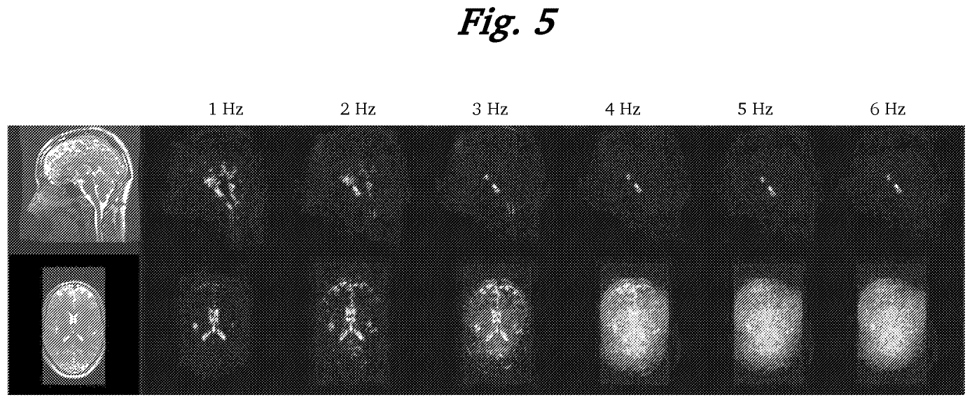

FIG. 5 shows the normalized temporal standard deviation maps of the amplified (3D aMRI) movies for different temporal frequency bands (1-6 Hz), demonstrating that motion information exists within the 1-4 Hz band. The data was amplified with an amplification parameter of 30 with a Gaussian window with σ=5. Motion was extracted using the first two levels of the steerable pyramid. Coherent motion exists mainly in the 1-4 Hz frequency band.

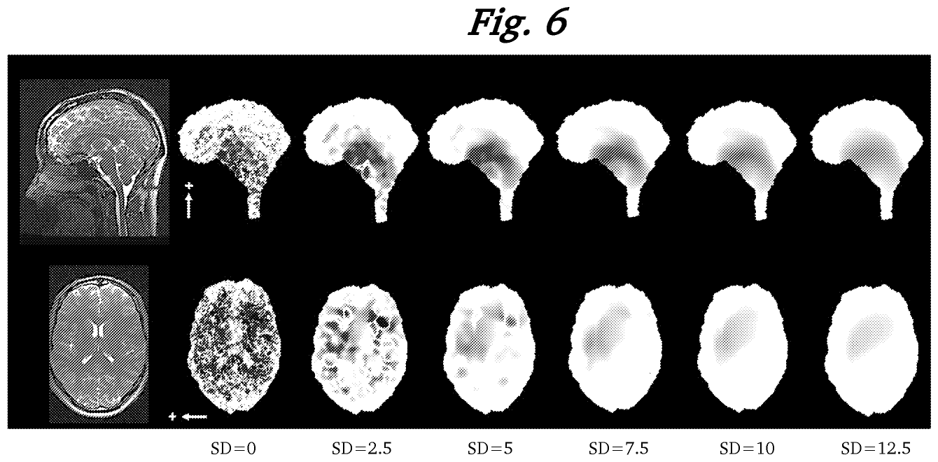

FIG. 6 depicts the extracted motion field by 3D q-aMRI for varying Gaussian windows (o ranging from 0-12.5). The estimated field profile is almost unchanged for σ>5. For these reasons, the hyperparameters were chosen as aforementioned. The figure shows pulsatile brain motion in the sagittal (S/I direction) and axial (L/R direction) for different standard deviation sizes of the Gaussian window. The Gaussian window reduces the noise level in the estimated motion field. For standard deviations larger than σ=5, the estimated motion field is smooth and generally remains constant.

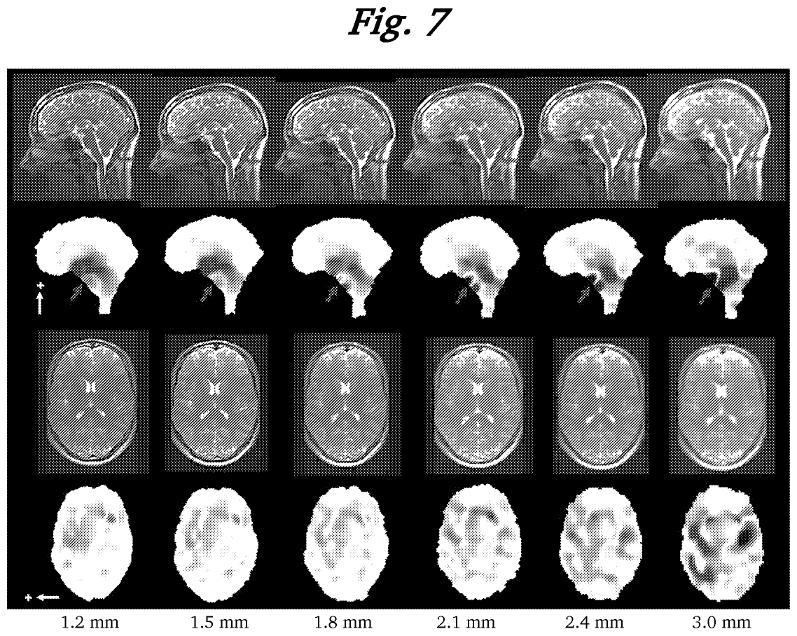

FIG. 7 depicts the extracted motion field by 3D q-aMRI for different isotropic voxel sizes (1.2-3.0 mm³). The extracted motion field remains consistent up to an isotropic voxel size of 1.8 mm³. The arrows in the sagittal plane point to the basal artery, which exhibits apparent motion (larger than 1.5 pixels), and which based on the phantom simulations will result in an error in the estimated motion field. The figure shows pulsatile brain motion in the sagittal (S/I direction) and axial (L/R direction) for different isotropic spatial resolutions. As can be seen, the technique can robustly estimate the motion field for different image resolutions (up to 1.8 mm³ isotropic voxel size).

### In Vivo Analysis and Repeatability Study

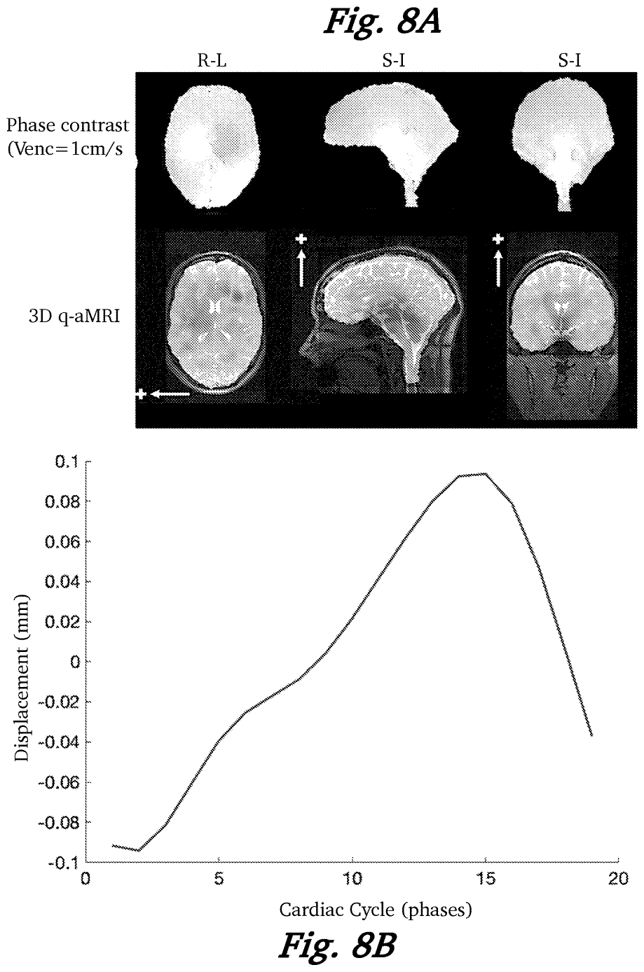

FIG. 8A shows an image comparison between PC-MRI (top) and 3D q-aMRI (bottom) for sagittal (S/I direction=), coronal (S/I direction), and axial (L/R direction) planes. The estimated field captures the relative brain tissue deformation over time and the physical change in shape of the ventricles by the relative movement of the surrounding tissues. In both 3D q-aMRI and cine PC-MRI, the general characteristics of brain motion were found to be similar. In both sequences, the predominant tissue displacement was in the cranial-caudal direction in the sagittal and coronal planes, and expanding/contracting motion in the axial plane, with the largest brain tissue displacement occurring around the midbrain, cerebellar tonsils, brainstem, and hypothalamus. Minimal displacement occurred in the frontal lobe, parietal lobe, occipital lobe, temporal lobe, and posterior cerebellum.

FIG. 8B depicts the voxel displacement profile (throughout the cardiac cycle) in the out-of-the-plane direction through the cerebral aqueduct as captured by 3D q-aMRI. This motion/flow profile is consistent with other literature. The figure shows the extracted flow/motion profile through the cerebral aqueduct as extracted by 3D q-aMRI. Note that the graph seen here is normalized, but the actual CSF flow values reported were an order of magnitude higher than the 3D q-aMRI flow profile.

The repeatability and reproducibility of the voxel displacement brain motion throughout the cardiac cycle was validated in five different brain regions. The results indicate high repeatability across the time points within each subject, with similar motion patterns but different magnitudes across all subjects.

The clinical potential of the method was also validated using 3D q-aMRI data of healthy control subjects and subjects with mild cognitive impairment (MCI) due to Alzheimer's disease (AD).

### DISCUSSION

This work introduces a novel 3D quantitative aMRI (3D q-aMRI) post-processing technique that enables the visualization and quantification of the sub-voxel pulsatile brain motion in physical units. The technique is based on solving the optical flow equation over the phase coefficients of the 3D complex steerable pyramid decomposition, and is an extension of the 3D aMRI technique.

The phantom simulations suggest that when properly tuned, 3D q-aMRI is capable of quantifying motion at a scale greater than 0.005 of a pixel size (the theoretical limit) for an SNR as low as 25. This is achieved while maintaining a high correlation (0.95), low error (13%), and a relatively small error distribution tail (99% of the error values fall below 30%). However, it is important to note that, in practice, there are likely confounding factors such as field inhomogeneity, partial volume effects, and various sources of noise, which were not comprehensively represented in the phantom simulation. These factors will likely limit 3D q-aMRI's ability to accurately estimate motions at the order of 0.005 of a pixel size. Yet, as demonstrated in FIG. 8B, the motion profile observed in the cerebral aqueduct is continuous and smooth. This suggests that the technique has successfully detected coherent signal changes corresponding to voxel displacement at a scale of 0.01 pixel. The precise limit boundary will need to be determined through real phantom experiments.

A noteworthy observation from the phantom simulation is that the technique performs very accurately, even for very low contrast data, such as what is seen in the brain ventricles. As long as some level of texture exists in the data and the voxels exhibit subtle signal changes corresponding to motion, the technique appears capable of accurately estimating the voxel displacement field-even in the presence of considerable noise (low SNR). This claim comes with the condition that in a small neighborhood, the motion field remains relatively constant—as this factor influences the technique's accuracy. Thankfully, this assumption seems valid for the human brain. It worth mentioning that some texture almost always exists in the in vivo data. For example, the lateral ventricles, which supposed to contain only CSF (same T1 and T2 property, and as such the same contract) contain some texture.

The study's in vivo validation demonstrates that 3D q-aMRI successfully quantifies (in the form of a voxel displacement field) the observed signal in 3D aMRI. The technique hyperparameters were tuned empirically according to the phantom simulation observations. We observed that most of the motion exists in the first two levels of the 3D steerable pyramid. This suggests that the pulsatile brain motion is composed of local deformations rather than global translation (rigid body motion). In addition, most of the motion is concentrated within the 1-4 Hz temporal band. This suggests that the cardiac input, which is periodic, contains higher harmonics in addition to the main heart rate (fundamental harmonic) and/or that the brain, as a nonlinear system, is excited by higher harmonic modes in response to the cardiac input. In addition, we noticed that for a Gaussian filter with σ>5, the estimated motion field profile is almost unchanged. This observation helps to determine a threshold for how much smoothing is needed to remove noise without over smoothing the estimated field. Finally, we determined the range of image isotropic resolution (1.2-1.8 mm³) for which the estimated motion field remains relatively consistent. Larger motion displacement was observed in the low-resolution data. This is likely to be due predominantly to errors in low resolution images which are plagued by partial volume effects. Our analysis here emphasizes the importance of standardizing 3D q-aMRI for clinical applications, as various parameters, including image resolution and dimension, Gaussian filter (o value), and temporal filtering frequency band, have an impact on the extracted brain motion field (mainly the amplitude). Notice that other true quantitative methods such as DENSE is also subject to variation in tissue displacement estimation as a function of image resolution.

It is worth mentioning that, like many other MRI techniques, 3D q-aMRI is prone to rigid body motions, which can be categorized into coherent and non-coherent motion. Non-coherent motion results in spatial blurring in the cine MRI data, which in turn results in an error in the estimated voxel displacement field. Coherent motion, such as bulk head motion induced by blood pulsation, is detectable by 3D q-aMRI and can be corrected by subtracting the estimated voxel displacement field in the skull (which is assume to be static) from the estimated displacement field

It should be noted that 3D q-aMRI estimates motion accurately up to 1.5 pixels. When dealing with heavily pulsating vessels like the basilar artery, which can move through several pixels in a cine dataset, these dynamic structures can introduce errors in the estimated motion field. This underscores the importance of thoroughly examining both the raw and amplified data in conjunction with the displacement field maps.

Previous research has illustrated the intricate nature of the brain's biomechanics, showing the interplay between the CSF and the pulsatile brain motion. The general characteristics of pulsatile brain motion observed in 3D q-aMRI resemble those in other studies. The predominant displacement of tissue and fluid occurs in the cranial-caudal direction in the sagittal and coronal planes. Additionally, the axial plane shows expansion/contraction motion, and the most significant tissue displacement is concentrated around the midbrain, brainstem, cerebellar tonsil, and hypothalamus regions. Due to the reduced perfusion of brain tissue toward the cranium boundary, minimal displacement is observed in the frontal lobe, parietal lobe, occipital lobe, temporal lobe, and posterior cerebellum.

It is noteworthy that our choice of the 3D q-aMRI technique's hyperparameters resulted in a motion/flow profile through the cerebral aqueduct, which matched results previously reported. While this is a promising finding that supports the current choice of the hyperparameters used here, the 3D q-aMRI flow profile was an order of magnitude (mm/s) smaller than that reported in the literature. This is an interesting finding; while 3D q-aMRI is not directly measuring CSF flow (as does PC-MRI), it may provide complementary information to flow about the tissue compliance. It seems like the cerebral aqueduct pulsatile motion may result from the pressure of the CSF flowing through it, and that regional brain displacement and CSF flow may be tightly coupled. This important observation suggests that 3D q-aMRI may be capable of detecting the CSF pulsatile flow profile in various brain compartments, such as the subarachnoid and perivascular space. Again, while the flow profile itself is not quantifiable, it could lead to an important method of indirectly assessing CSF homeostasis, and further testing should be done to confirm this.

The in vivo validation of our study highlights the potential for 3D q-aMRI to be used as a reliable quantitative tool in clinical studies for generating pulsatile brain motion heat maps, which may help to assess disease-induced biomechanical differences. Our study observed the most substantial brain motion occurs in the superior-inferior direction, consistent with previous studies. This motion is likely attributed to the pulsatile nature of the CSF flow, which is strongly influenced by the cardiac cycle.

Additionally, our results demonstrate that 3D q-aMRI is both repeatable and reproducible. The analysis suggests significant differences between within to across subjects, which shows the potential to detect individual differences, even in this healthy volunteer cohort. It is also worth noting that heart rate exerts a significant influence on the estimated motion magnitude. We believe this is a direct physiological consequence of cardiac pulsatility. Our data is cardiac-gated, and motion is assumed to result from blood flow and pulsations transferred through the larger arteries within a cardiac cycle. Hence, any alterations in the amplitude or frequency of these pulsations, such as natural heart rate variations, are likely to impact the timescale available for transferring these pulsations carried by pulsatile blood flow.

It is also important to recognize that while 3D q-aMRI demonstrated reliable detection of sub-voxel brain motion, other MRI techniques (CSPAMM, PC-MRI, and DENSE MRI) may also be capable of detecting this type of motion. With its high temporal resolution, high spatial resolution, good tissue contrast, short scan time, and ability to accurately capture three-dimensional brain movements, 3D q-aMRI is a practical clinical alternative that can also reliably capture brain motion in three-dimensions. For example, the total acquisition time for a DENSE scan in a 3T scanner with 2.2 mm isotropic resolution is 16.5 min. However, it is crucial to highlight that 3D q-aMRI estimates the voxel displacement field based on the signal intensity temporal changes in bSSFP cine MRI. While the measured signal in DENSE and PC-MRI corresponds to the genuine spin displacement, in bSSFP, there are other physiological sources that may impact the signal intensity and cause 3D q-aMRI to interpret it as motion (like any other optical flow method), even if they do not necessarily correlate to true displacement. As such the method should not be considered as a true displacement quantification method like DENSE-MRI. There is no doubt that the extracted signal is highly correlated with tissue displacement, but further work is also necessary to characterize the true physiology behind the 3D q-aMRI signal variation.

The preliminary data from our study suggests that 3D q-aMRI holds potential in identifying disease-induced biomechanical differences and can serve as a diagnostic tool for early signs of neurodegenerative disorders. In all cases, abnormal brain motion was detected compared to healthy controls. In the sagittal plane, incoherent abnormal (differ from the usual piston-like) motion was seen in the lateral ventricles, corpus callosum, aqueduct, and 4^{rd} ventricle. In the axial plane, abnormal motion was detected mainly around the lateral ventricles, which exhibited diffuse and asymmetric motion compared to healthy controls. Some cases were also associated with general reduction in brain motion. Our preliminary study suggests that abnormal pulsatile brain motion might correlate with changes associated with cognitive decline, however, it is important to note that these findings are based on a small number of healthy volunteers and further studies with larger and more diverse populations are necessary to validate these results and establish the clinical utility of 3D q-aMRI.

### CONCLUSION

This disclosure provides a 3D quantitative aMRI (3D q-aMRI) post-processing technique capable of visualizing and quantifying voxel displacement pulsatile brain motion in physical units. Applied to regular 3D cine data, the method enjoys short scan time and fast processing time, making it a practical tool for clinical use. 3D q-aMRI is both repeatable and reproducible, although it is imperative to consider the effects of different physiological confounders and acquisition parameters when interpreting 3D q-aMRI data. Nevertheless, 3D q-aMRI could provide valuable insights into neurological pathologies and their impact on the brain's biomechanical response, making it a potentially useful tool for research and diagnosis.

1. A method for magnetic resonance imaging comprising:
(a) performing by a magnetic resonance imaging (MRI) apparatus an MRI imaging scan of a brain to produce 3D MRI video of the brain;
(b) processing the 3D MRI video to amplify and quantify in the 3D MRI video a sub-voxel motion field induced by physiological brain motion by solving an optical flow equation over phase coefficients of a 3D complex linear steerable pyramid decomposition of the 3D MRI video.

2. The method of claim 1 wherein the 3D MRI video comprises 3D cardiac-gated cine bSSFP data, 3D bSSFP cine data, cine Gradient-Spoiled SSFP data, Cine RF Spoiled SSFP data, or SSFP-Echo data.

3. The method of claim 1 wherein phases of the 3D complex linear steerable pyramid decomposition are used to produce magnification and quantification of the 3D MRI video, wherein the phases are separated from the amplitude component, band-passed, processed using a conventional 3D aMRI technique to produce motion magnification, and processed through a quantification technique that solves the optical flow equation over the phases using a weighted least squares objective function over the various orientation and scales of the 3D steerable pyramid decomposition.

4. A method for magnetic resonance imaging comprising:
(a) performing by a magnetic resonance imaging (MRI) apparatus an MRI imaging scan of a brain to produce 3D MRI videos of a brain;
(b) processing the 3D MRI videos to produce a sub-voxel motion field of the brain induced by physiological motion, wherein the processing comprises:
i. generating a 3D complex linear steerable pyramid decomposition of the 3D MRI videos into multiple scales and orientations;
ii. temporally filtering phase coefficients of the 3D complex linear steerable pyramid decomposition;
iii. solving an optical flow equation over the phase coefficients of the 3D complex linear steerable pyramid decomposition to produce the sub-voxel motion field in the form of voxel displacement maps;
(c) displaying the sub-voxel motion field.

5. The method of claim 4 wherein processing the 3D MRI videos is performed by the magnetic resonance imaging (MRI) apparatus.

6. The method of claim 4 wherein processing the 3D MRI videos is performed by a device distinct from the magnetic resonance imaging (MRI) apparatus.

7. The method of claim 4 wherein the physiological motion is periodic physiological motion.

8. The method of claim 4 wherein the physiological motion is non-periodic physiological motion.

9. The method of claim 4 wherein the 3D MRI videos are steady state free precession (SSFP) sequence MRI data.

10. The method of claim 9 wherein the 3D MRI video comprises 3D cardiac-gated cine bSSFP data, 3D bSSFP cine data, cine Gradient-Spoiled SSFP data, Cine RF Spoiled SSFP data, or SSFP-Echo data.
